(19) United States
(12) Patent Application Publication
(10) Pub. No.: US 2015/0284338 A1
(43) Pub. Date: Oct. 8, 2015

(54) INDUSTRIAL PROCESS FOR THE SYNTHESIS OF PYRIMIDINYL-PIPERAZINE DERIVATIVES

(71) Applicant: RICHTER GEDEON NYRT., Budapest (HU)

(72) Inventors: Krisztina Szavicskó, Budapest (HU); György Domány, Budapest (HU); László Czibula, Budapest (HU); Gizella Bartáné Szalai, Budapest (HU); László Dobay, Budapest (HU); Éva Werkné Pap, Budapest (HU)

(73) Assignee: Richter Gedeon Nyrt., Budapest (HU)

(21) Appl. No.: 14/646,112

(22) PCT Filed: Nov. 20, 2013
(86) PCT No.: PCT/IB2013/060265

§ 371 Date: May 20, 2015

(30) Foreign Application Priority Data
Nov. 21, 2012 (HU) P1200675

(51) Int. Cl. C07D 239/48

(52) U.S. Cl. CPC C07D 239/48

(57) ABSTRACT

Our invention relates to trans-(4-{4-[2-(4-amino-cyclohexyl)-ethyl]-piperazin-1-yl}-5,6-dichloro-pyrimidin-2-yl)-methylamine dihydrochloride monohydrate, the process for the synthesis of this compound, the new (4,5-dichloro-6-piperazin-1-yl-pyirimidin-2-yl)-methylamine monohydrochlorid used as starting material in this synthesis, as well as the industrial process for the synthesis of trans-N-(4- {2-[4-(5,6-dichloro-2-methyl-amino-pyirimidin-4-yl-)piperazin-1-yl]-ethly}cyclohexyl-propionamide starting from trans-(4-{4-[2-(4-amino-cyclohexyl)-ethyl]-piperazin-1-yl}-5,6-dichloro-pyrimidin-2-yl)-methylamine dihydrochloride monohydrate.

### SUMMARY OF THE INVENTION

Our invention relates to trans-(4-{4-[2-(4-amino-cyclohexyl)-ethyl]-piperazin-1-yl}-5,6-dichloro-pyrimidin-2-yl)-methylamine dihydrochloride monohydrate of formula (III), the process for the synthesis of this compound, the new (4,5-dichloro-6-piperazin-1-yl-pyirimidin-2-yl)-methylamine monohydrochloride of formula (IV) used as starting material in the synthesis of compound of formula (III), as well as the industrial process for the synthesis of trans-N-(4-{2-[4-(5,6-dichloro-2-methyl-amino-pyirimidin-4-yl)-piperazin-1-yl]-ethyl}cyclohexyl-propionamide of formula (I) starting from compound of formula (III).

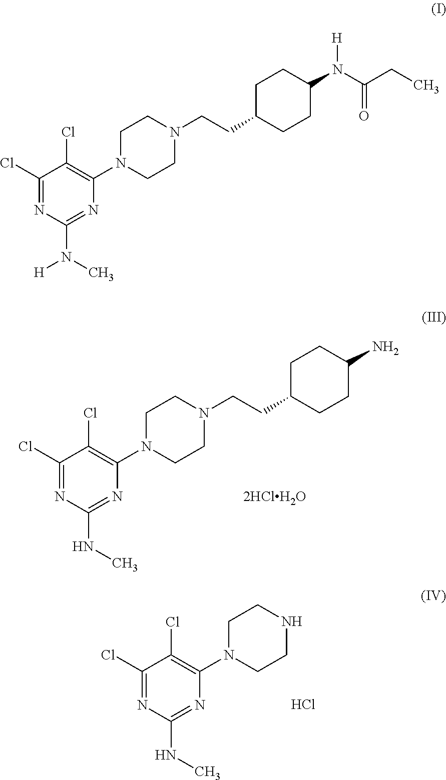

### BACKGROUND OF THE INVENTION

Trans-(4-{4-[2-(4-amino-cyclohexyl)-ethyl]-piperazin-1-yl}-5,6-dichloro-pyirimidin-2-yl)-methylamine trihydrochloride of formula (II) is an intermediate in the synthesis of compound of formula (I) described in the Hungarian patent application HU20070000269 of Richter.

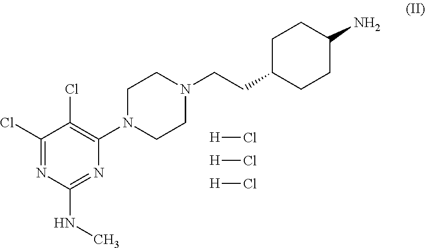

The synthesis of compound of formula (II) is described in examples 1-4 of the Hungarian patent application HU20070000269. According to process “A” of the patent application HU20070000269 only the acetamide derivative is described in the examples starting from trans-(4-{4-[2-(4-amino-cyclohexyl)-ethyl]-piperazin-1-yl}-5,6-dichloro-pyrimidin-2-yl)-methylamine trihydrochloride of formula (II) and the yield of this process is only 70%. (The examples do not describe the experiments carried out with the propionic acid derivative.)

### DETAILED DESCRIPTION OF THE INVENTION

Our aim was to elaborate an industrial process for the synthesis of compound of formula (I), which results in the product in good yield and acceptable purity without column chromatography.

Surprisingly it was found, that in the process using chromatography for the purification the quality and the purity of the intermediate trans-(4-{4-[2-(4-amino-cyclohexyl)-ethyl]-piperazin-1-yl}-5,6-dichloro-pyrimidin-2-yl)-methylamine trihydrochloride of formula (II) is responsible for the only 70% yield. Therefore our further aim was to synthesize trans-(4-{4-[2-(4-amino-cyclohexyl)-ethyl]-piperazin-1-yl}-5,6-dichloro-pyrimidin-2-yl)-methylamine or a derivative thereof in such purity from which the final product can be obtained in high yield and purity without using chromatography for the purification.

During our experiments we found, that if instead of (4,5-dichloro-6-piperazin-1-yl-pyrimidin-2-yl)-methylamine dihydrochloride—described in the example 3 of the Hungarian patent application HU20070000269—the formerly unknown (4,5-dichloro-6-piperazin-1-yl-pyrimidin-2-yl)-methylamine monohydrochloride of formula (IV)—synthesized under different conditions and described in example 6 of the present invention was reacted with trans-2-{1-[4-(tert-butoxycarbonyl)-amino]-cyclohexyl}-ethyl-methanesulfonate—described in example 3 of the Hungarian patent application HU20080000763—then the trans-(4-{2-[4-(5,6-dichloro-2-methylamino-pyrimidin-4-yl)-piperazin-1-yl]-ethyl}-cyclohexyl)-carbamic acid tert-butyl-ester was obtained in high purity.

Surprisingly it was found furthermore that if the protecting group of the intermediate synthesized as described above is removed under aqueous-acidic conditions (preferably in the presence of a strong inorganic acid or an organic sulfonic acid) then a so far unknown derivative, the trans-(4-{4-[2-(4-amino-cyclohexyl)-ethyl]-piperazin-1-yl}-5,6-dichloro-pyrimidin-2-yl)-methylamine dihydrochloride monohydrate of formula (III) can easily be obtained in high yield and purity.

Surprisingly it was found that treatment of the intermediate (III) obtained as described above with propionic acid halogenide or propionic anhydride afforded the compound of formula (I) in a yield of more than 95% and in high purity without purification by chromatography. The acylation is preferably carried out with a propionic acid derivative in an apolar solvent in the presence of a base. Preferably propionic acid anhydride, chloride or bromide can be applied as propionic acid derivative and dichloromethane or toluene as solvent. The acylation is preferably carried out between 0 and 25° C., more preferably between 20 and 25° C. Tertiary amines are used as base, preferably triethylamine, N-methyl-morpholine or pyridine.

Our invention relates to trans-(4-{4-[2-(4-amino-cyclohexyl)-ethyl]-piperazin-1-yl}-5,6-dichloro-pyrimidin-2-yl)-methylamine dihydrochloride monohydrate of formula (III), the process for the synthesis of this compound, the new (4,5-dichloro-6-piperazin-1-yl-pyirimidin-2-yl)-methylamine monohydrochlorid of formula (IV) used as starting material in the synthesis of compound of formula (III), as well as the new process for the synthesis of trans-N-(-4{2-[4-(5,6-dichloro-2-methyl-amino-pyirimidin-4-yl)-piperazin-1-yl]ethyl}cyclohexyl-propionamide of formula (I) starting from compound of formula (III) which affords the desired compound in high yield and purity even on an industrial scale as compared to the process described in the previous Hungarian patent application HU20070000269.

The detailed synthesis of compound of formula (I) is described in examples 3, 4 and 5.

### EXAMPLES

The invention is illustrated by the following, not limiting examples.

### Example 1

### (trans-(4-{2-[4-(5,6-dichloro-2-methylamino-pyrimidin-4-yl)-piperazin-1-yl]ethyl}-cyclohexyl)-carbamic acid tert-butyl ester)

To a stirred slurry of 48 g (0.15 mol) of trans-2-{1-[4-(N-tert-butoxycarbonyl)-amino]-cyclohexyl}-ethyl methanesulfonate in 800 ml of acetonitrile 44.8 g (0.15 mol) of (4,5-dichloro-6-piperazin-1-yl-pyrimidin-2-yl)-methylamine monohydrochloride and subsequently 71.8 g (0.56 mol) of finely grind potassium carbonate were added and stirring was continued at reflux temperature for 15-17h. Thereafter the stirred reaction mixture was cooled to 45-50° C., 900 ml of water was added and the mixture was cooled to room temperature. Stirring was continued at this temperature for further 1.5 h. The precipitated product was filtered, washed with water till neutrality, thereafter it was stirred in a solution of 400 ml of water and 7 ml of concentrated hydrochloric acid for 2 h at 20-25° C. The slurry was filtered, washed with 100 ml of water in three portions and with 10 ml of cold acetonitrile. The product was dried till constant weight at maximum 50° C.

Yield: 57.7 g (79%). Mp.: 200-202° C.

### Example 2

### (trans-(4-{4-[2-(4-amino-cyclohexyl)-ethyl]-piperazin-1-yl}-5,6-dichloropyrimidin-2-yl)-methylamine dihydrochloride monohydrate

To a stirred slurry of 30 g of (trans-(4-{2-4-(5,6-dichloro-2-methylamino-pyrimidin-4-yl)-piperazin-1-yl1-ethyl}-cyclohexyl)-ethyl)-carbamic acid tert-butyl ester obtained according to example 1 in a mixture of 300 ml of isopropyl alcohol and 30 ml of ion exchanged water 51 ml of concentrated HCl was added at 25-30° C. The slurry was stirred at 55-60° C., thereafter the reaction mixture was slowly cooled to 2-5° C. and the slurry was stirred for 1 h, filtered and washed with 60 ml of a 10:1 mixture of isopropyl alcohol and water in two portions. The product was dried at maximum 50° C. till constant weight.

Yield: 26.1 g (90%) water-content: 4.1% (Karl-Fischer)

### Example 3

### (trans-N-(4-{2-[4-(5,6-dichloro-2-methyl-aminopyrimidin-4-yl)-piperazin-1-yl]-ethyl}cyclohexyl-propionyl amide)

To a stirred slurry of 7.2 g (0.0150 mol) of trans-(4-{4-[2-(4-aminocyclohexyl)-ethyl]-piperazin-1-yl}-5,6-dichloro-pyrimidin-2-yl)-methylamine dihydrochloride monohydrate in 90 ml of dichloromethane 5 g (0.0495 mol) of triethylamine was added at 10-12° C. Thereafter 2.55 g (0.0195 mol) of propionic anhydride was added and the reaction mixture was stirred at 20-25° C. for 3 h. The precipitate was filtered, mixed with 100 ml of ion exchanged water in two portions, filtered and washed with 30 ml of ion exchanged water in two portions. The obtained product was dried till constant weight at maximum 30° C.

Yield: 6.4 g (96.2%) Mp.: 248-250° C.

### Example 4

### (trans-N-(4-{2-[4-(5,6-dichloro-2-methylamino-pyrimidin-4-yl)-piperazin-1-yl]-ethyl}cyclohexyl-propionyl amide)

To a stirred slurry of 9.0 g (0.0188 mol) of trans-(4-{4-[2-(4-aminocyclohexyl)-ethyl]-piperazin-1-yl}-5,6-dichloropyrimidin-2-yl)-methylamine dihydrochloride monohydrate in 90 ml of dichloromethane 9.6 g (0.0948 mol) of triethylamine was added at 23-25° C. and the mixture was cooled to 0-5° C. Thereafter a solution of propionyl chloride (3.48 g, 0.0375 mol) in dichloromethane (15 ml) was added and the reaction mixture was stirred at 0-5° C. for 2 h. The precipitated product was filtered, stirred with 100 ml of ion exchanged water in two portions for 1 h, filtered and washed with 40 ml of ion exchanged water in two portions. The obtained product was dried till constant weight at maximum 50° C.

Yield: 7.7 g (92%) Mp.: 248-250° C.

### Example 5

### (trans-N-(4-{2-[4-(5,6-dichloro-2-methyl-aminopyrimidin-4-yl)-piperazin-1-yl]-ethyl}cyclohexyl-propionyl amide)

To a stirred slurry of 4.76 g (0.01 mol) of trans-(4-{4-[2-(4-aminocyclohexyl)-ethyl]-piperazin-1-yl}-5,6-dichloro-pyrimidin-2-yl)-methylamine dihydrochloride monohydrate in 50 ml of dichloromethane 5.1 g (0.05 mol) of triethylamine was added at 23-25° C. and the mixture was cooled to 0-5° C. Thereafter a solution of propionyl bromide (2.75 g, 0.02 mol) in dichloromethane (7 ml) was added and the reaction mixture was stirred at 2-5° C. for 5 h. After completion of the reaction the product was filtered, mixed with 100 ml of ion exchanged water in two portions, the mixture was stirred at room temperature for 1 h, filtered and washed with 40 ml of ion exchanged water in two portions. The obtained product was dried till constant weight at maximum 50° C.

Yield: 3.3 g (75%) Mp.: 248-250° C.

### Example 6

### (4,5-dichloro-6-piperazin-1-yl-pyrimidin-2-yl)-methylamine monohydrochloride

Under nitrogen to a stirred solution of 15.1 g (0.04 mol) of [4,5-dichloro-6-(4-tert-butoxycarbonyl-piperazin)-1-yl-pirimidin-2-yl]methylamine in 150 ml of ethyl acetate 25 ml of concentrated HCl was added, then the mixture was warmed to 45-50° C. and stirred at this temperature for 2 h. After completion of the reaction the mixture was cooled to 0-(−1)° C. and left at this temperature for 2 h. The precipitated product was filtered, washed with 2×20 ml of cold (0° C.) ethyl acetate. The obtained product was dried till constant weight at maximum 50° C.

Yield: 10.6 g (89%)

1. Trans-(4-{4-[2-(4-amino-cyclohexyl)-ethyl]-piperazin-1-yl}-5,6-dichloro-pyrimidin-2-yl)-methylamine dihydrochloride monohydrate of formula (III)

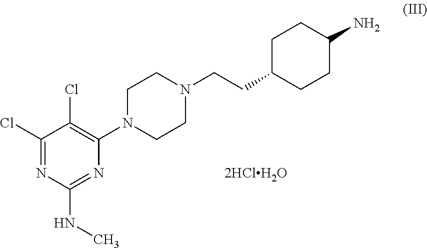

2. (4,5-dichloro-6-piperazin-1-yl-pyirimidin-2-yl)-methylamine monohydrochlorid of formula (IV).

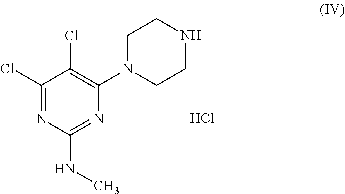 [SMILES: CNc1nc(Cl)c(Cl)c(N2CCNCC2)n1 ; Cl]

3. Process for the synthesis of trans-(4-{4-[2-(4-amino-cyclohexyl)-ethyl]-piperazin-1-yl}-5,6-dichloro-pyrimidin-2-yl)-methylamine dihydrochloride monohydrate of formula (III) characterized by
a) reacting the compound of claim 2, (4,5-dichloro-6-piperazin-1-yl-pyirimidin-2-yl)-methylamine monohydrochlorid of formula (IV), with trans-2-{1-[4-(tert-butoxycarbonyl)-amino]-cyclohexyl}-ethyl-methanesulfonate;
b) then removing the protecting group of the obtained trans-(4-{2-[4-(5,6-dichloro-2-methyl-amino-pyrimidin-4yl)-piperazin-1-yl]-ethyl}-cyclohexyl)-carbamic acid tert-butyl ester under aqueous-acidic conditions.

4. Process for the synthesis of trans-N-(4-{2-[4-(5,6-dichloro-2-methyl-amino-pyirimidin-4-yl)-piperazin-1-yl]-ethyl}cyclohexyl-propionamide of formula (I) characterized by acylating the compound of claim 1, trans-(4-{4-[2-(4-amino-cyclohexyl)-ethyl]-piperazin-1-yl}-5,6-dichloro-pyrimidin-2-yl)-methylamine dihydrochloride monohydrate of formula (III), with a propionic acid derivative in the presence of a base, in an apolar solvent, at 0-25° C.

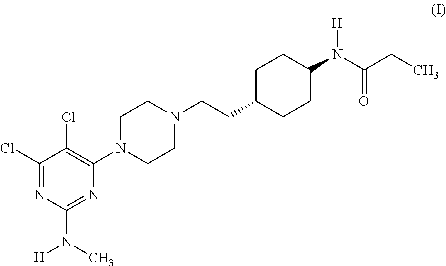

5. The process according to claim 4 characterized by using propionic anhydride, propionyl chloride or propionyl bromide as propionic acid derivative.

6. The process according to claim 4 or claim 5 characterized by using dichloromethane or toluene as apolar solvent.

7. A process according to any of claims 4-6 characterized by carrying out the reactions between 20 and 25° C.

8. A process according to any of claims 4-7 characterized by using tertiary amines as base.

9. A process according to any of claims 4-8 characterized by using triethyl amine as base.
